(19) United States
(12) Patent Application Publication
(10) Pub. No.: US 2022/0395680 A1
(43) Pub. Date: Dec. 15, 2022

(54) DEVICE FOR ANCHORING A CARDIAC PUMP AND ASSEMBLY FOR FITTING A CARDIAC PUMP PROVIDED WITH SUCH AN ANCHORING DEVICE

(71) Applicant: FINEHEART, PESSAC (FR)

(72) Inventors: Jérémy Collas, Bordeaux (FR); Stéphane Garrigue, Begles (FR); Emmanuel Bougere, Pessac (FR)

(21) Appl. No.: 17/755,807

(22) PCT Filed: Nov. 13, 2020
(86) PCT No.: PCT/FR2020/052083

§ 371 Date: May 9, 2022

(30) Foreign Application Priority Data
Nov. 15, 2019 (FR) 1912799

(51) Int. Cl. A61M 60/863; A61M 60/178; A61M 60/216; A61M 60/411

(52) U.S. Cl. CPC A61M 60/863; A61M 60/178; A61M 60/216; A61M 60/411

(57) ABSTRACT

A medical device for anchoring a cardiac pump in an opening of a ventricular wall of a heart, including a connector to be fitted to the ventricular wall. The connector includes a connection flange and a support skirt. The skirt is to be placed outside the heart when the flange is joined to the ventricular wall. The connector defines a first opening for a part of the body of an insert to pass through. The insert is rigid and defines a second opening for the body of the pump to pass through. The insert includes a distal end that is to be placed inside the heart and a proximal end that is to be placed outside the heart when the insert is joined to the connector. At least one element locks/unlocks the insert in position with respect to the connector when the insert has been introduced into the connector.

### CROSS-REFERENCE TO RELATED APPLICATIONS

This application is a National Stage of International Application No. PCT/FR2020/052083, having an International Filing Date of 13 Nov. 2020, which designated the United States of America, and which International Application was published under PCT Article 21(2) as WO Publication No. 2021/094698 A1, which claims priority from and the benefit of French Patent Application No. 1912799, filed on 15 Nov. 2019, the disclosures of which are incorporated herein by reference in their entireties.

### BACKGROUND

### Field

The present disclosure relates to a device for anchoring a cardiac pump in an opening of a ventricular wall of a beating heart or of a non-beating heart benefiting from an extracorporeal circulation.

It also relates to an assembly for fitting a cardiac pump incorporating such an anchoring device.

### BRIEF DESCRIPTION OF RELATED DEVELOPMENTS

Heart failure (HF) is a pathological state in which a patient's heart is unable to provide a rate of blood flow necessary for the metabolic needs of the body.

It is known to treat heart failure by implanting a ventricular assist device (VAD), which is an artificial cardiac pump.

This mechanical pump does not replace the heart, which continues to function, but provides assistance to the weakened ventricle so as to increase the rate of blood flow in a manner suited to the needs of the individual.

This assistance may be temporary while waiting for a graft to become available for performing a heart transplant.

However, a significant proportion of patients will not receive such a graft, either because they cannot be candidates for such a transplant, for example because of severe heart failure, or because no suitable graft is available for these patients.

In that case, ventricular assistance is used as the intended solution, which is to say that the artificial cardiac pump is implanted for the long term.

These cardiac pumps are therefore the subject of intensive research aimed at improving the quality of life of patients presenting with heart failure, since these pumps can remain active for several months to several years.

Many advances have been made, and ventricular assist devices are known today which are more compact and quieter and provide an increased service life.

The implantable cardiac pumps of the prior art are thus typically equipped with an integrated electric motor to ensure their operation, the speed of rotation of the pump providing the force necessary for circulating the blood from the weakened ventricle to the circulatory system.

Systems for implanting such pumps in an opening of a ventricular wall are also known.

These implantation systems generally comprise a tubular portion, at the ends of which are placed, or formed, collars which are intended to be each pressed against an opposite face of the ventricular wall after introduction of the tubular portion into an opening made in this ventricular wall with a coring device.

For example, there is known a hollow tubular portion at the proximal end of which is placed a first flange and at the distal end of which is placed a membrane self-expandable between a first configuration, called the deformed configuration, in which this membrane has a tubular shape, and a second configuration, called the initial configuration, in which it defines a second flange. This initial configuration is achieved when this distal end is introduced inside a patient's heart.

Such collars thus make it possible to maintain the hollow tubular portion in position, which then defines an orifice passing through the ventricular wall.

At the end, placed outside the heart, of this tubular portion, a cardiac pump is inserted which, once installed, ensures that the blood present in the ventricle returns to the circulatory system.

Although giving good results, these implantation systems can be further improved.

In particular, they are difficult to implement and to place on a beating heart. They require great dexterity on the part of the practitioners performing the intervention in order to ensure the success of the latter, and this is a source of stress.

There is therefore a need for an anchoring device whose original design makes it possible to overcome the disadvantages of the prior art that have been set out above.

### SUMMARY

The present disclosure aims to overcome the disadvantages of the prior art and to meet the above demands by making available a device for anchoring a cardiac pump, which device is simple in terms of its design and its operating mode and reduces the complexity of the surgical intervention while being very reliable.

The present disclosure also relates to such an anchoring device ensuring complete sealing between the inside and the outside of the heart.

The present disclosure also relates to an assembly for fitting a cardiac pump in place, having such an anchoring device, this assembly making it possible to reduce the patient's loss of blood during the surgical intervention and to enhance the patient's recovery.

To this end, the present disclosure relates to a device for anchoring a cardiac pump in an opening of a ventricular wall of a heart, comprising a connector intended to be mounted on this ventricular wall.

According to the present disclosure,
this connector comprises a joining flange and a support skirt, or tubular projection, extending from this joining flange, this support skirt being intended to be placed outside the heart when the joining flange is joined to the ventricular wall, this connector defining a first opening for the passage of a part of the body of an insert,
this insert being entirely rigid and defining a second opening for the passage of the body of the cardiac pump, this insert comprising a distal end intended to be placed inside the heart and a proximal end intended to be placed outside the heart, when this insert is mounted on the connector rigidly joined to the ventricular wall, and
at least one element for positionally locking/unlocking the insert with respect to the connector, when this insert has been introduced into the connector.

Such a device for anchoring a cardiac pump in the opening of a ventricular wall of a heart is also known as an anchoring ring, or fixing device, of a cardiac pump.

It is advantageously made entirely of a biocompatible, non-toxic and sterile material.

Such a device is particularly suitable for an intervention on a beating heart or a non-beating heart benefiting from an extracorporeal circulation.

This anchoring device can thus consist simply of a connector able to be mounted on a heart and defining an orifice, an insert intended to be carried by this connector, by having a part of its body passing through an opening made in the cardiac wall, this opening being surrounded by this connector, and one or more elements ensuring the locking in position of the insert with respect to the connector when this insert has been mounted on this connector rigidly joined to this ventricular wall.

Advantageously, such an anchoring device provides greater convenience for the surgical team during the operation, since this anchoring device is particularly easy to fit in place. It also ensures enhanced safety for the patient, since complete sealing is achieved between the inside and outside of the patient's heart, of the one-way valve type with two or three leaflets, a diaphragm or flap valve.

In the context of the present disclosure, the term “proximal” means the position closest to the healthcare professional, or the practitioner, while the term “distal” is to be understood here as meaning farthest away from this professional. In other words, the distal end of an element is the end that would be the first to be engaged in a patient's heart, while its proximal end would be the last end to be engaged therein.

According to a particular embodiment of this anchoring device, with this connector comprising a reinforcing flange placed at its proximal end, the insert also has a reinforcing flange, these reinforcing flanges being intended to be pressed against each other when the insert is mounted on the connector.

According to another embodiment of this anchoring device, the locking/unlocking element is a clamping element passing laterally through the reinforcing flange of the connector in order to clamp the body of the insert, or a fixing element intended to join together the reinforcing flanges of the connector and of the insert.

For example, this clamping element can be a screw.

As this screw has an external thread, the through-orifice of the reinforcing flange has a thread complementary to the screw thread, in order to ensure the engagement and movement of this screw, and also the required sealing.

Alternatively, this clamping element could be a bayonet clamping system, the grooves of which would be located on the inner surface of the support skirt of the connector.

According to another embodiment of this anchoring device, the rigid insert has a tubular portion, the longitudinal dimension of which allows the distal end of this insert to be placed inside the heart cavity when the insert is mounted on the connector.

According to another embodiment of this anchoring device, the connector and/or the insert comprise one or more grooves, each of these receiving a seal, preferably an O-ring seal.

According to another embodiment of this anchoring device, this insert comprises one or more sealing elements in order to ensure a sealed join between this insert and the pump body intended to be inserted into the second opening.

According to another embodiment of this anchoring device, the joining flange is made of a material making it possible to promote the growth or the attachment of cells, in order to reinforce its join with the ventricular wall.

This material is also able to be sutured or stapled.

Purely for illustrative purposes, this joining flange is made of Dacron® or polytetrafluoroethylene (PTFE).

According to another embodiment of this anchoring device, the distal end of the insert is chamfered in order to facilitate its insertion through an orifice formed in the ventricular wall.

According to another embodiment of this anchoring device, the insert has a fixing element for locking the cardiac pump body in position when the latter has been introduced into the second opening.

The present disclosure also relates to an assembly for fitting a cardiac pump in place on a heart, comprising an anchoring device as described above and a removable sheath having a non-return valve, or one-way valve, this sheath being intended to be mounted on at least the proximal end of the insert in order to prevent any loss of blood during the insertion or removal of a tool, such as a coring tool, or of the cardiac pump.

Upon the removal of a coring tool, for example, this one-way valve closes and becomes sealed again. By way of example, it is a self-repairing membrane, also called a self-sealing membrane.

Advantageously, this removable sheath is made of a flexible material so that it can sealingly cover at least the proximal end of the insert.

This sheath can be inserted around the proximal ends of the connector and of the insert in order to cover these elements.

Advantageously, this sheath is made of silicone or any other flexible biocompatible material, such as polyurethane (PU) or polyether ether ketone (PEEK).

Alternatively, this sheath could be rigid by being made, for example, of titanium or of polyether ether ketone (PEEK). It would then have a single thread or a bayonet clamping system in order to be fixed on the support skirt of the connector and/or the reinforcing flange of the insert.

Preferably, this removable sheath has a purge chamber, the non-return valve being placed at the proximal end of this sheath.

This purge chamber is advantageously dimensioned to accommodate the head of a coring tool.

Thus, while the proximal end of this sheath has a non-return valve, its distal end is open so as to be able to be introduced and surround at least the proximal end of the insert.

Advantageously, the removable sheath has a purge orifice for the evacuation of the air, a closure element making it possible to close this orifice in order to ensure the necessary seal.

The present disclosure also relates to a kit for treating heart disease, comprising an anchoring device as described above and a cardiac pump comprising a casing configured to be introduced through the second orifice defined by the insert, while ensuring a seal with one or more sealing elements carried by the insert.

Preferably, this cardiac pump has a turbine, movable in rotation with respect to the casing, and an inductor.

### BRIEF DESCRIPTION OF THE DRAWINGS

Other advantages, aims and particular features of the present disclosure will become clear from the following description, which is provided for explanatory purposes and is non-limiting and in which reference is made to the accompanying drawings, where:

FIG. 1 is a perspective view of a connector of an anchoring device according to a particular embodiment of the present disclosure;

FIG. 2 is a front view of the insert of the anchoring device of FIG. 1;

FIG. 3 is a perspective view, seen from the proximal end of the insert, of the anchoring device of FIG. 1, showing sealing elements and a through-orifice in the flange to allow the passage of a clamping screw;

FIG. 4 is an X-ray of a heart on which are mounted an anchoring device of FIG. 1 and a cardiac pump for assisting the latter;

FIG. 5 shows a fitting assembly in which a cardiac pump is introduced.

### DETAILED DESCRIPTION

The drawings and the description below contain, for the most part, elements of certain character. They may therefore not only serve for better understanding the present disclosure, but also contribute to its definition, where appropriate.

First, it will be noted that the figures are not to scale.

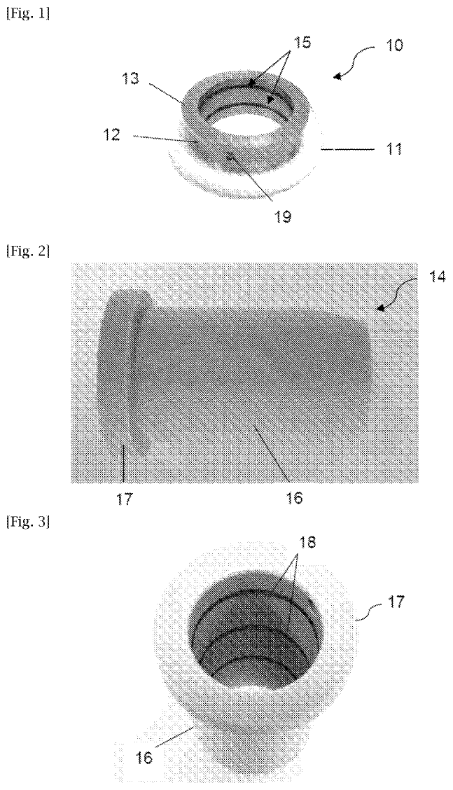

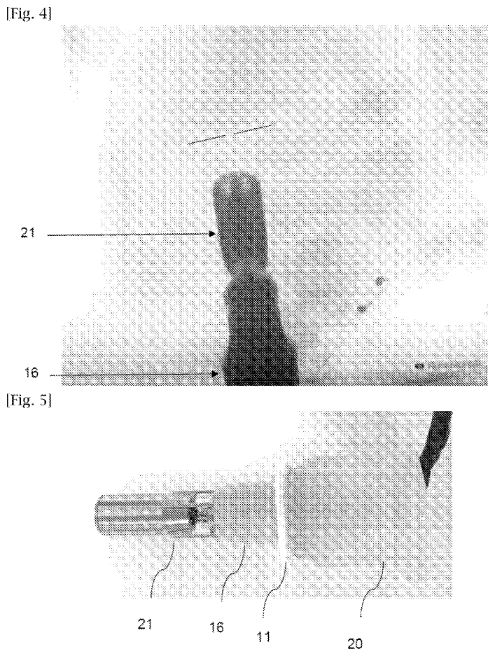

FIGS. 1 to 3 schematically illustrate an anchoring device according to a particular embodiment of the present invention.

This anchoring device has a connector 10 which can be mounted on a ventricular wall of a heart of a patient whose heart failure is to be treated.

This connector 10 comprises a joining flange 11, a tubular projection 12 extending axially from this joining flange, and a support flange 13.

This joining flange 11 is here intended to be fixed to the ventricular wall by suturing. It is advantageously made of a material facilitating the growth or the attachment of cells, in order to reinforce its join with the ventricular wall.

The tubular projection 12 and the support flange 13 are made in one piece from a rigid non-thrombogenic material, for example of titanium.

This tubular projection 12 and the support flange 13 are intended to be placed outside the heart when the joining flange 11 is joined to the ventricular wall, this connector defining a first opening for the passage of a part of the body of an insert 14.

Sealing elements 15 comprising O-rings are carried by the tubular projection 12 in order to seal the join between the connector 10 and the insert 14.

This insert 14, which is entirely rigid, has a tubular part 16 and a reinforcing flange 17. Only the tubular part 16 of this insert 14 is intended to be introduced in part through the ventricular wall and to be in contact with this ventricular wall.

This insert 14 is here made of polyether ether ketone (PEEK), but it could also be made of titanium.

The outer surface of the insert 14 can have a surface relief, such as one or more meshes, in order to promote the adherence of the myocardial tissue and to promote endothelialization, that is to say tissue covering of the insert inside the ventricle.

The free end of this tubular part 16, or the distal end of this insert 14, is chamfered in order to facilitate its introduction through an opening made in the ventricular wall by means of a coring tool (not shown).

This hollow insert 14 defines a second opening for the passage of the body of a cardiac pump. Grooves receiving 0-rings 18 are placed in the inner surface of the insert 14 delimiting the second opening, in order to cooperate with the outer surface of the casing of a cardiac pump in such a way as to provide the required sealing. These O-rings 18 could also be placed on the outer surface of the insert 14, for example on the tubular part 16 at the foot of the reinforcing flange 17.

The distal end of this insert 14 is intended to be placed inside the heart while its proximal end, defined by the reinforcing flange 17, is intended to be placed outside the heart when it is mounted on connector 10.

The tubular projection 12 of the connector 10 has a through-orifice and a clamping screw 19 inserted in this orifice in order to allow the body of the insert 14 to be positionally locked/unlocked with respect to the connector 10, when this insert 14 has been introduced into the connector 10 through the first opening.

This particularly simple anchoring device makes it possible, by virtue of its anchoring in an opening made in the ventricular wall of a heart, to receive and support the body of a cardiac pump used to assist this failing heart.

To ensure the manipulation of a cardiac pump 21, and in particular its introduction into the anchoring device, a removable sheath 20 having a non-return valve, or one-way valve, is used to close the second opening defined by the insert 14.

The non-return valve permits the introduction and the removal of the body of a cardiac pump or of a coring tool with minimal loss of blood.

This sheath 20 is made of a flexible material, for example of silicone, in order to permit its introduction around support flange 13 and reinforcing flange 17. This sheath 20 is configured to cover these elements in a sealed manner.

It has a purge chamber (not shown), the size of which makes it possible in particular to accommodate the head of the coring tool after cutting the ventricular wall in order to form an opening.

1. A medical device for anchoring a cardiac pump in an opening of a ventricular wall of a heart, comprising a connector intended to be mounted on said ventricular wall, characterized in that:
this connector comprises a joining flange and a support skirt extending from this joining flange, this support skirt being intended to be placed outside the heart when the joining flange is joined to the ventricular wall,
said medical anchoring device also comprises an insert, this connector defining a first opening for the passage of a part of the body of this insert,
this insert being entirely rigid and defining a second opening for the passage of the body of the cardiac pump, this insert comprising a distal end intended to be placed inside the heart and a proximal end intended to be placed outside the heart when this insert is mounted on the connector, and
at least one element for positionally locking/unlocking the insert with respect to the connector, when this insert has been introduced into the connector.

2. The medical device for anchoring the cardiac pump as claimed in claim 1, characterized in that, with this connector comprising a reinforcing flange placed at its proximal end, the insert also has a reinforcing flange, said reinforcing flanges being intended to be pressed against each other when the insert is mounted on the connector.

3. The medical device for anchoring the cardiac pump as claimed in claim 2, characterized in that the locking/unlocking element is a clamping element passing laterally through the reinforcing flange of the connector in order to clamp the body of the insert, or a fixing element intended to join together the reinforcing flanges of the connector and of the insert.

4. The medical device for anchoring the cardiac pump as claimed in claim 1, characterized in that the rigid insert (14) has a tubular portion, the longitudinal dimension of which allows the distal end of this insert to be placed inside the heart cavity when the insert is mounted on the connector.

5. The medical device for anchoring the cardiac pump as claimed in claim 1, characterized in that the connector and/or the insert comprise one or more grooves, each of these receiving a seal, and in that the insert comprises one or more sealing elements to ensure a sealed join between this insert and the pump body intended to be inserted into the second opening.

6. The medical device for anchoring the cardiac pump as claimed in claim 1, characterized in that the joining flange is made of a material making it possible to promote the growth or the attachment of cells, in order to reinforce its join with the ventricular wall.

7. The medical device for anchoring the cardiac pump as claimed in claim 1, characterized in that the distal end of the insert is chamfered in order to facilitate its insertion through an orifice formed in the ventricular wall.

8. The medical device for anchoring the cardiac pump as claimed in claim 1, characterized in that the insert has a fixing element for locking the cardiac pump body in position when the latter has been introduced into the second opening.

9. An assembly for fitting a cardiac pump in place on a heart, comprising the medical device for anchoring the cardiac pump as claimed in claim 1 and a removable sheath having a non-return valve, or one-way valve, this sheath being intended to be mounted on at least the proximal end of the insert in order to prevent any loss of blood during the insertion or removal of a tool or of a cardiac pump.

10. The assembly for fitting the cardiac pump as claimed in claim 9, characterized in that this removable sheath is made of a flexible material so that it can sealingly cover at least the proximal end of the insert.

11. The assembly for fitting the cardiac pump as claimed in claim 9, characterized in that the removable sheath has a purge chamber, the non-return valve being placed at the proximal end of this sheath.

12. The assembly for fitting the cardiac pump as claimed in claim 1, characterized in that the removable sheath has a purge orifice for the evacuation of the air, a closure element making it possible to close this orifice.

13. A kit for the treatment of heart disease, comprising the medical device for anchoring the cardiac pump as claimed in claim 1 and a cardiac pump comprising a casing configured to be introduced through the second opening defined by the insert, while ensuring a seal with one or more sealing elements carried by the insert.

14. The kit as claimed in claim 13, characterized in that the cardiac pump has a turbine, movable in rotation with respect to the casing, and an inductor.
